(19) United States
(12) Patent Application Publication
(10) Pub. No.: US 2011/0021871 A1
(43) Pub. Date: Jan. 27, 2011

(54) Laparoscopic surgical instrument

(76) Inventor: Gerry Berkelaar, Norwell, MA (US)

Correspondence Address: John M. Brandt, 60 Thaxter St., Hingham, MA, 02043, US

(21) Appl. No.: 12/804,651

(22) Filed: Jul. 27, 2010

Related U.S. Application Data
(60) Provisional application No. 61/271,765, filed on Jul. 27, 2009.

(51) Int. Cl. A61B 1/00

(52) U.S. Cl. 600/104

(57) ABSTRACT

An articulating surgical instrument for laparoscopic, endoscopic, and natural orifice translumenal endoscopic surgical procedures consisting of a separable combination of a reusable operational handpiece section and a separable reusable or disposable articulation section. The handpiece section which remains external to the patient incorporates various control and command devices and assemblies, while the articulation section is composed of various linkages, rods, and cables which transmit positioning and operating commands from the handpiece to end effectors positioned at the distal tip of the articulation section within the patient. Improved positioning and operating configurations for the end effectors suitable for the invention as well as the general class of these instruments are also disclosed.

### CROSS REFERENCE TO RELATED APPLICATIONS

This application is based on the disclosure of Provisional Application Ser. No. 61/271,765 filed Jul. 27, 2009 which is hereby incorporated by reference.

### BACKGROUND OF THE INVENTION

1. Field of the Invention

The invention resides in the field of surgical apparatus and more particularly relates to instruments for performing laparoscopic, endoscopic, and natural orifice translumenal endoscopic surgery.

2. Description of the Prior Art

Devices of the type to which the invention is directed are well known in the prior art. Generally classed as laparoscopic or endoscopic surgical instruments, their purpose is to perform surgical procedures within the body through relatively small incisions which allow the insertion of small surgical tools or end effectors mounted on elongated shafts which are manipulated or articulated to position the surgical tool and then are further operated to achieve the desired surgical procedure. The articulation, positioning and activation of the tools are all controlled by a corresponding handpiece located outside the body.

Illumination and viewing of the internal surgical site is accomplished by, for example, an additional device of the same general type employing a light source and miniature-imaging device such as a TV camera.

In contrast to the prior art, the present invention provides improved devices and assemblies for operating an end effecter, rotating the end effecter about the longitudinal axis of the articulation portion of the instrument, as well as for a control and operation system for articulating or bending the tip of the articulation portion and an attached end effecter.

The handpiece and articulation sections may also be separable in order to prolong the life of one or the other components of the instrument, to use multiple articulation sections with different end effecters with a single handpiece, and to provide for the prevention of reuse of a particular articulation portion or section.

Examples of prior art instruments which are illustrative of and relate to the invention are shown in U.S. Pat. Nos. 5,454,827, Aust et al., a distal tip articulation mechanism; 5,578,052, Koros et al., a separable handpiece and articulation section; and 5,860,995, Berkelaar, an end effecter articulation assembly.

### SUMMARY OF THE INVENTION

The invention may be summarized as a laparoscopic surgical instrument composed of an operational handpiece and an elongated articulation section having a flexible bending portion. These two components may be permanently connected, or separate and connectable. The mechanism joining the two components may optionally include an arrangement for preventing reuse of the articulation section by, for example, destroying or altering a portion of the connecting linkage to preclude the possibility of reconnection.

The handpiece contains the control mechanisms for positioning and operating a surgical tool or end effector disposed at the tip of the articulation section. These controls may include a knob or lever for rotating a flexible link or rod connected to the end effecter for rotating the end effecter, means to laterally move the same or an additional link to activate the end effector, such as a scissors, and hand or motor operated means to simultaneously pull and release appropriate cable or wire connections to the bending portion to alter or articulate its position or orientation to any point within a spherical space along the longitudinal axis of the instrument.

Electronic controls for achieving the articulation of the bending portion through the use of motor driven gear assemblies are also disclosed.

The articulation section, in general, an elongated hollow shaft with a flexible bending portion, contains the above mentioned wires, links, and/or rods as well as suitable protective sheaths or coverings. The shaft, providing the main support structure for the end effecter, may be comprised in part of a series of segments or links which will move in relation to one another to produce the articulation.

These and other features and advantages of the invention will be more fully understood from the following description of the preferred embodiment in conjunction with the accompanying drawings.

### DESCRIPTION OF THE DRAWINGS

FIG. 1 is a perspective frontal (distal) view of the preferred embodiment of the invention;

FIG. 2 is a perspective rear (proximal) view of the preferred embodiment of the invention;

FIG. 3 is a perspective frontal view of a separated portion of the embodiment of FIG. 1;

FIG. 4 is a perspective frontal view of an additional separated portion of the embodiment of FIG. 1;

FIG. 5 is a cross-sectional view of a portion of FIG. 2 along line A-A;

FIG. 6 is a side view of a component of FIG. 1;

FIG. 7 is a partial cross-sectional view of the component of FIG. 6;

FIG. 8 is a perspective view of a component of FIG. 5;

FIG. 9 is a perspective view of the component of FIG. 5;

FIG. 10 is a perspective detailed view of an internal assembly of a component of FIG. 3;

FIG. 11 is an exploded perspective view of a component of FIG. 4;

FIG. 12 is a side view of a component of FIG. 11;

FIG. 13 is a perspective view of a portion of the assembly of FIG. 10;

FIG. 14 is a side view of a portion of the assembly of FIG. 10;

FIG. 15 is a side view of a portion of the assembly of FIG. 14;

FIG. 16 is a detailed perspective view of the component of FIG. 4;

FIG. 17 is a perspective view of a component of FIG. 9; and

FIG. 18 is an additional perspective view of a portion of FIG. 9

### DESCRIPTION OF THE PREFERRED EMBODIMENT

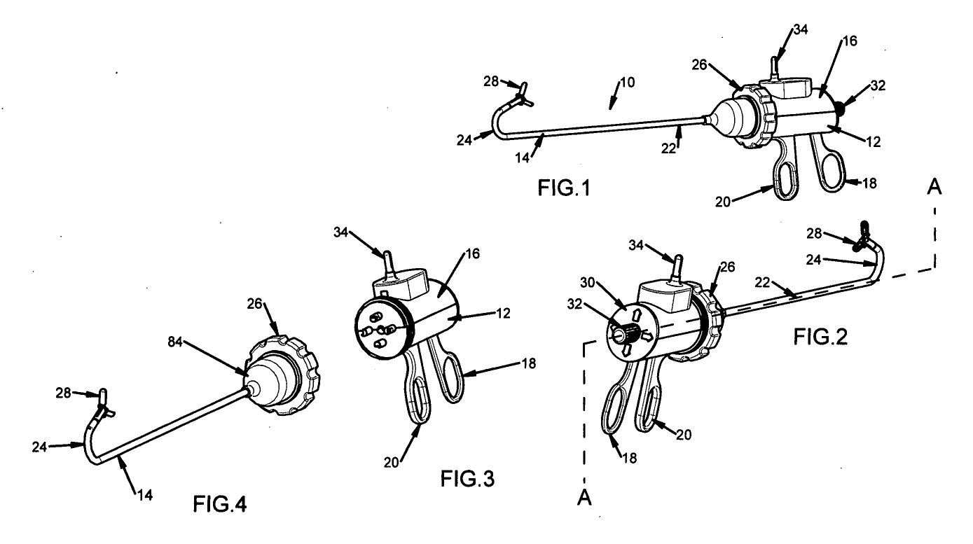

FIG. 1 is a perspective frontal view of the preferred embodiment of the laparoscopic instrument 10 of the invention for performing multiscopic surgery that is capable of holding, grasping, cutting, hooking, manipulating, burning, and coagulating tissue along a non linear trajectory. This instrument is a combination of an operational handpiece 12 and an articulation section 14. A variety of different articulation sections and their associated end effectors are used to perform different surgical tasks.

Handpiece 12 has an outer case 16 surrounding and supporting control mechanisms to be described in detail below, a trigger handle 18, and an opposing finger grip 20. Articulation section 14 consists of an elongated hollow shaft 22, a flexible bending portion 24, and joining nut 26 for attaching handpiece 12 and articulation section 14. An end effecter or surgical tool is attached or mounted on tip 24.

FIG. 2 is a rearward or proximal view of the instrument of FIG. 1 further showing an articulation control backplate 30 mounted on case 16 for controlling the position or orientation of bending portion 24. A rotary thumb actuator 32 for rotating end effecter 28 is also mounted on case 16 in conjunction with backplate 30. An electrical connection post 34 for enabling cauterization procedures is further positioned atop case 16.

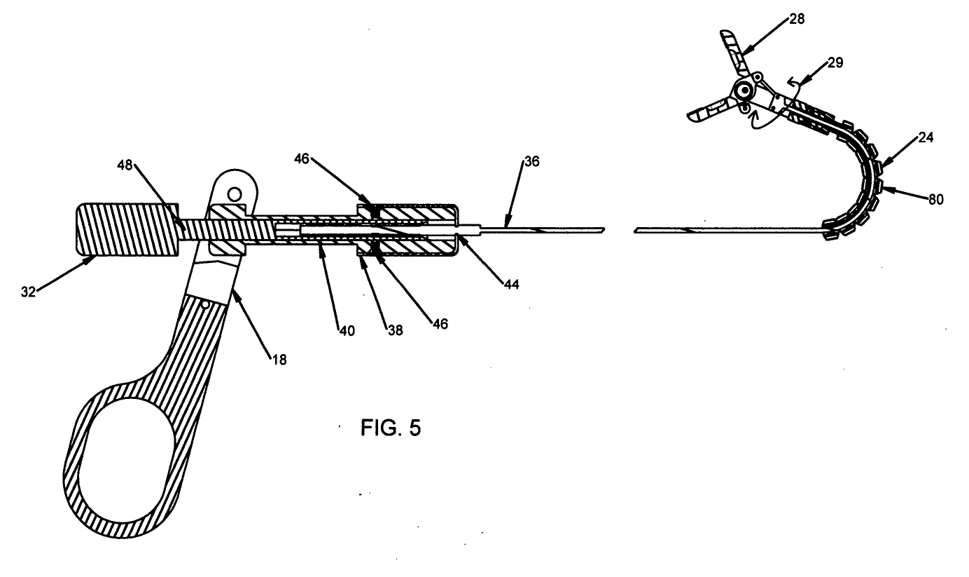

FIGS. 3 and 4 illustrate handpiece 12 and articulation section 14 separated from one another. FIGS. 1-4 above present an overview of the general configuration of the invention, the components and operation of which will now be described in accordance with FIGS. 5-18 in more particular detail as follows.

FIG. 5 shows, in cross-sectional format, the components and control assembly which provide for the axis rotation of articulation section 14, more specifically, a selected end effecter 28 disposed at the distal tip of bending portion 24 as indicated by motion arrow 29. This is accomplished by the rotation of pushrod 36 disposed in shaft 22 as controlled or moved by thumb actuator 32 mounted on handpiece 12.

Pushrod 36 is cylindrical with a non-round shaped tip that is pushed through the center of the teeth 38 that are mounted on lock assembly 40 disposed in handpiece 12. Pushrod 36 is aligned with the center of teeth 38 of the lock assembly and extends through the center of the assembly. Pushrod 36 continues further until it engages and is positioned inside rotary thumb actuator 32 where it mechanically mates against a non-round inside wall. Pushrod 36 does not bottom out against the proximal end of actuator 32. Instead, it is allowed clearance to slide back and forth axially inside actuator 32. The total linear distance in which pushrod 36 can slide is determined by the travel needs of bending portion 24 as it bends from a straight line to its maximum deflected position. Also compensated for is the travel distance of end effecter 28 as it opens and closes as or if required. Both events are able to occur simultaneously. Rotary thumb actuator 32 is fixed to the control backplate 30. Actuator 32 rotates radially about the center axis but is fixed in position axially to backplate 30 and does not move forward or backward.

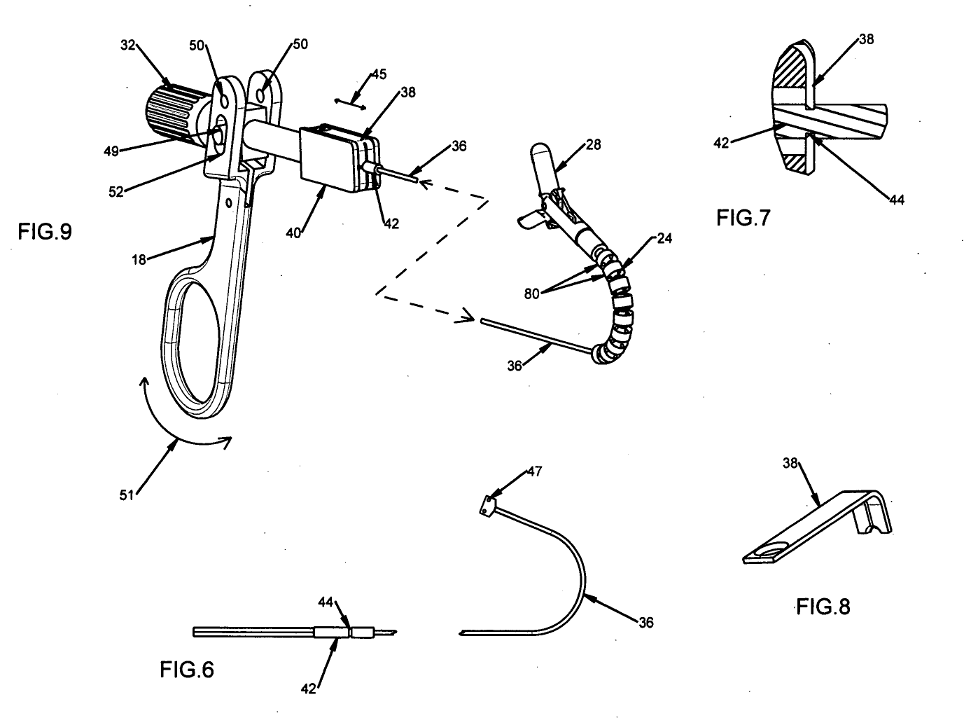

As shown in FIGS. 6 and 7, the proximal end 42 of pushrod 36, which fits within handpiece 12 and is of a larger diameter than the much longer distal portion, has a cylindrical notch 44. The backside of notch 44 engages the front side of teeth 38 mounted on lock assembly 40 by locator screws 46. Teeth 38 provide a clamping force against pushrod segment 42 within notch 44 thereby keeping the entire pushrod fixed within lock assembly 40.

Therefore, when rotary thumb actuator 32 is turned in either radial direction, end effecter 28 mounted on pushrod endplate 47 will turn in compliance with the rotation of the actuator. Actuator 32 thereby drives the rotation of end effecter using by way of pushrod 36.

As described above, flexible metal teeth 38, as shown in perspective in FIG. 8, are secured to lock assembly 40 with two locator screws 46, one for each tooth. Lock assembly 40 travels forward and in reverse and is controlled by the trigger handle 18 closing against finger grip 20. The front side of each tooth pushes against the backside of the cylindrical notch and drives the pushrod forward. End effecter 28 then closes. When the trigger handle 18 is pulled in the opposite direction away from the finger grip 20, lock assembly 40 travels in reverse, the opposite side of notch 44 is pushed, and end effecter opens 28.

As shown in FIG. 7, the backsides of the teeth are also beveled to allow the disengagement of the pushrod when handpiece 12 and articulation section 14 are separated. The cylindrical nature of notch 44 allows pushrod 36 to be rotated radially while simultaneously fixing it into position axially.

As further illustrated by FIG. 9, lock assembly 40 is shuttled forward and in reverse, proximally and distally, as indicated by arrow 45, by the action, arrow 51, of trigger handle 18. Lock assembly 40 also travels axially over the distal portion of rotary actuator 32, using the rotary actuator as a guide. Lock assembly 40 is also connected to trigger 18 by means of two outboard pins 49 (one not shown) that may be a molded feature of assembly 40. Trigger 18 is further connected to handpiece 12 on swing pivots 50. The swing pivot is a molded feature of the handpiece 12. Engagement slots 52 in trigger handle 18 receive the outboard pins 49. When the trigger handle 18 is moved, outboard pins 49 impart the driving force to move lock assembly 40. Engagement slots 52 allow the required clearance necessary for the arc travel of trigger handle 18 without binding against assembly 40. The user places their thumb into the ring of trigger handle 18 and their fingers into the ring of finger grip 20. Closing the hand provides the force to shuttle the lock assembly 40 back and forth thereby manipulating end effecter 28.

The positioning of an end effecter at a desired orientation at a surgical site within the body can be provided by articulation and in this instrument is achieved by the use of four cables extending from the handpiece to the distal end of the bending portion where they are attached in an orthogonal array such that by withdrawing one cable toward the proximal end and simultaneously releasing it's corresponding opposite cable disposed directly across from the cable being withdrawn, the end effecter will move up or down, left or right, and toward or away from the surgeon depending upon which cable pair is selected for manipulation. This may be carried out manually by, for example, thumbwheel devices or preferably as described below by a motorized system using driving motors, appropriate gear systems and a hand operated control to select the desired angle and amount of articulation.

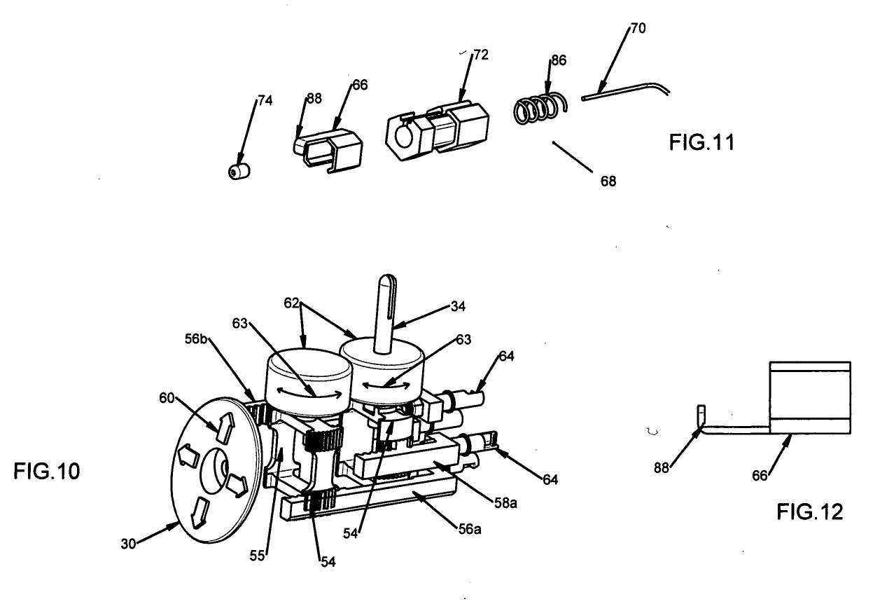

Referring to FIG. 10, there are two sets of two rotatable motion motor gears 54 mounted on motor gear frame 55 which drive two sets of associated linear motion gear rack pairs 56a and 56b and 58a and 58b all of which are mounted inside handpiece 12. When used in combination they cause articulation up and down and also articulation left and right. The resultant motion of articulation is not limited to up, down, left or right. The hand operated control, as indicated by control backplate directional arrows 60, allows for any angle between these positions. In this instrument the two motors convert rotary motion to linear motion using the gear drive that causes the end effecters to travel in a circular trajectory.

Two reversible motors 62, one each for manipulating each pair of opposing articulating cables, are wired to backplate 30 and installed in handpiece 12. Backplate 30 is mounted onto the back of handpiece 12 and is marked with the backplate directional arrows 60 that indicate direction of articulation. Each opposing arrow operates a switch that in turn activates one or the other of the motors in the appropriate direction as indicated by arrows 63. Backplate 30 is also wired to a remote motor power supply (not shown) that may be controlled by foot pedals set on the floor of an operating room and within easy reach of the surgeon.

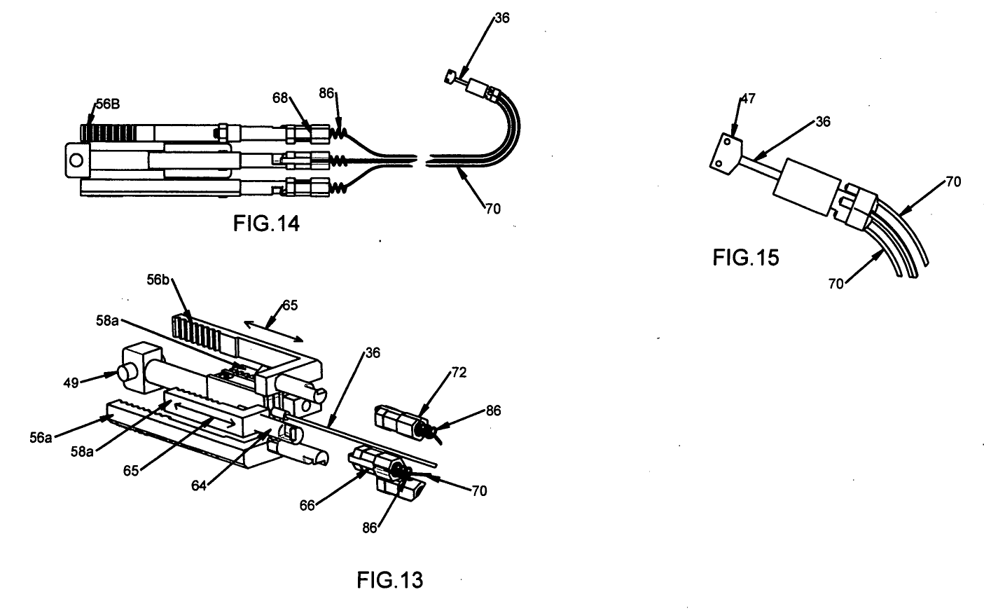

When pressure is applied to any arrow 60 on backplate 30, the corresponding motor gear turns in the corresponding direction. The appropriate gear rack will move either forward or in reverse, arrows 65, FIG. 13, depending upon which arrow is pressed. As a gear rack moves forward, it's opposite corresponding gear rack moves in the opposite direction, the motor gear teeth meshing with the gear rack teeth. When not energized, the motors are used as a positive locking mechanism for the entire assembly, that is, at rest, the gear racks cannot move.

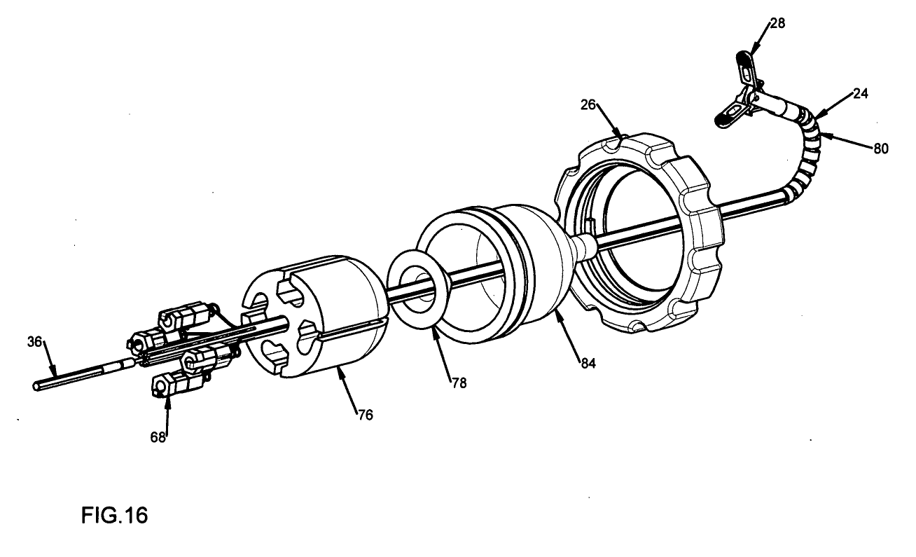

As shown in FIGS. 13 and 14, the distal ends of the gear racks are connected to connector pins 64. Connector pins 64 are shaped in a specific way that allows them to easily release away from opposing mating connector clips 66, FIG. 12, which are located in articulator section 14 shown in FIG. 16

Articulator section 14 connects to handpiece 12 to form the complete multiscopic surgical instrument. Articulator section includes two sets of opposing cable assemblies and a selected end effecter of choice. Each cable assembly 68, FIG. 11, is further comprised of a cable 70 assembled to a connector housing 72 by means of a crimp connector 74 which in turn is snapped onto a connector clip 66. Cable guides 76 and 78 are shown in FIG. 16. The two cable sets are paired and are arranged for articulation of bending portion 24 up and down and left and right. When a directional arrow 60 on control backplate 30 is pushed thereby activating one of the appropriate motors 62, a gear rack pulls on its mating cable assembly while the opposite mating cable assembly is released by the opposite gear rack to travel in the opposite direction. FIG. 15 shows the attachment of opposing cables 70 to the distal end of bending section 24.

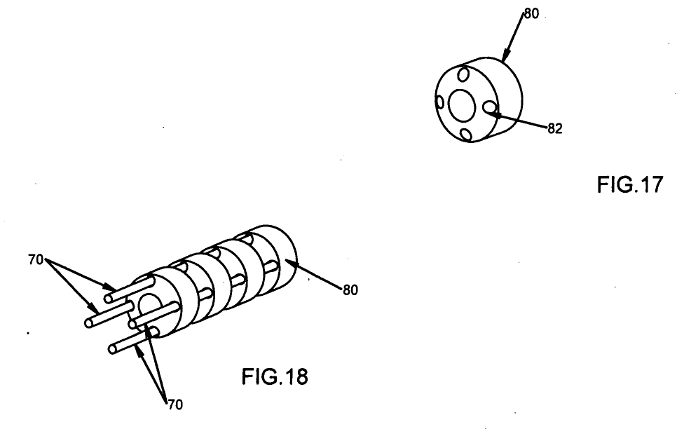

Articulation section 14 may be composed of a linked together plurality of individual segments 80 each having two sets of orthogonal ports 82 serving as cable guides, a section of which is shown in FIGS. 17 and 18.

When connecting articulator section 14 to handpiece 12, the first contact is made by pushrod 36 which extends beyond the base of the articulation section 14 bottom by a length that is long enough to snap the pushrod notch 44 against teeth 38 of the lock assembly 40. As pushrod 36 enters the center of lock assembly 40's teeth set, the four connector clips 66 simultaneously snap and lock onto the four connector pins 64. Pushrod 36 is longer than the extension of the connector clips 66 and enters handpiece 12 and the lock assembly before connector clips 66 are snapped into the four connector pins 64.

Articulation section 14 includes a trocar adapter 84 which engages and locks against handpiece 12. Compression springs 86 apply pressure against connector pins 64. The spring force ensures simultaneous constant pressure against all four of the Connector Pins allowing for simultaneous connection of the four connector pins to the four connector clips and additionally provides for slack removal. Articulation section 14 is finally securely joined to handpiece 12 by Nut 26.

To separate articulation section 14 and handpiece 12, nut 26 is unscrewed. As the nut is being unscrewed, the back wall of the nut presses against the back wall of trocar adapter 84 causing the two components, articulator section 14 and handpiece 12 to separate. The clips 66 in the articulation section are pulled at the same time but are stopped by the length of the cable. The continued travel of articulation section 14 causes the right angle clip extension 88 on the clip 66 to go past its yield point and straighten out. In this straightened condition, the four clips 66 cannot be used a second time and thereby rendering articulation section 14 unusable for continued use.

The scope of the invention is hereby defined by the following claims.

What is claimed is:

1. A laparoscopic surgical instrument comprising in combination the components of:
A. an articulation section, said articulation section comprising in combination:
i. an elongated shaft having a proximal end and a distal end and at least one passageway disposed along the entire length of said shaft;
ii. a flexible bending portion disposed at said elongated shaft distal end having a proximal end and a distal end and at least one passageway disposed along the entire length of said bending portion;
iii. at least one flexible control member having a proximal end and a distal end disposed in said passageways along the entire length of said shaft and said bending portion;
B. an end effecter mounting assembly attached to the distal end said control member; and
C. A handpiece connected to the proximal end of said articulation section, the proximal end of said shaft, and the proximal end of said flexible control member, said handpiece for moving and controlling said control member and said end effecter mounting assembly.

2. The surgical instrument of claim 1 further including an end effecter mounted on said end effecter mounting assembly

3. The surgical instrument of claim 2 wherein said end effecter is operated by said control member.

4. The surgical instrument of claim 3 wherein said end effecter is rotated by said control member.

5. The surgical instrument of claim 4 further including connecting means for connecting and disconnecting said handpiece and said articulation section whereby said instrument is separable into said articulation section and said handpiece.

6. The surgical instrument of claim 5 wherein said connecting means further includes means to prevent the reconnection of said articulation section and said handpiece upon disconnecting said articulation section and said handpiece.

7. A laparoscopic surgical instrument comprising in combination the components of:
A. an articulation section, said articulation section comprising in combination:
i. an elongated shaft having a proximal end and a distal end and at least one passageway disposed along the entire length of said shaft;
ii. a flexible bending portion disposed at said elongated shaft distal end having a proximal end and a distal end and at least one passageway disposed along the entire length of said bending portion;
iii. at least one flexible control member having a proximal end and a distal end disposed in said passageways along the entire length of said shaft and said bending portion;
B. an end effecter mounting assembly attached to the distal end said control member;
C. means for articulating said bending portion about a complete circular pattern with respect to the axis of said shaft, said means comprising a plurality of cables attached to said bending portion distal end, said cables disposed in said passageways along the entire length of said shaft and said bending portion; and
D. a handpiece connected to the proximal end of said articulation section, the proximal end of said shaft, the proximal end of said flexible control member, and the proximal end of said cables, said handpiece for controlling and moving said control member and said end effecter mounting assembly, said handpiece for further controlling and moving said cables and said bending portion.

8. The surgical instrument of claim 7 wherein said cables comprise four in number, two pairs each attached orthogonally about said bending portion distal end.

9. The surgical instrument of claim 8 wherein each of said cable pairs are simultaneously pulled and released to accomplish said articulation.

10. The surgical instrument of claim 9 further including two reversible rotary electric motors and two sets of linear motion gear racks operated by said motors disposed in said handpiece and wherein said cable pairs are attached one each to one each of said sets of gear racks, and wherein said cable pairs are simultaneously pulled and released by the operation of said motors and said gear racks.

11. The surgical instrument of claim 10 wherein said reversible motors are controlled by electrical switches mounted on the back of said handpiece.

12. The surgical instrument of claim 7 further including an end effecter mounted on said end effecter mounting assembly.

13. The surgical instrument of claim 12 wherein said end effecter is operated by said control member.

14. The surgical instrument of claim 13 wherein said end effecter is rotated by said control member.

15. The surgical instrument of claim 14 further including connecting means for connecting and disconnecting said handpiece and said articulation section whereby said instrument is separable into said articulation section and said handpiece.

16. The surgical instrument of claim 15 wherein said connecting means further includes means to prevent the reconnection of said articulation section and said handpiece upon disconnecting said articulation section and said handpiece.
